(19) United States
(12) Patent Application Publication
(10) Pub. No.: US 2026/0355491 A1
(43) Pub. Date: Nov. 12, 2026

(54) DEVICE AND METHOD FOR CONNECTING TUBES MADE OF PLASTIC

(71) Applicant: LMB SOFT DOO NIS, Palilula (Nis) (RS)

(72) Inventor: Klaus Jentsch, Palilula (Nis) (RS)

(21) Appl. No.: 19/134,739

(22) PCT Filed: Nov. 30, 2023
(86) PCT No.: PCT/EP2023/083746

§ 371 Date: Jun. 1, 2025

(30) Foreign Application Priority Data
Dec. 2, 2022 (DE) 10 2022 132 053.7

(51) Int. Cl. A61M 39/14; A61J 1/10; A61J 1/16; F16L 47/02

(52) U.S. Cl. CPC A61M 39/143; A61J 1/10; A61J 1/16; F16L 47/02

(57) ABSTRACT

A device for connecting first and second tubes includes a first clamp with jaws, a second clamp with jaws, a first separation unit, and a second separation unit. Each separation unit has an anvil and a blade for separating the first and second tubes at a respective separation point. A moving unit moves at least one of the first and second clamps such that separated ends of the first and second tubes are brought into axial alignment and then into abutment. The jaws and/or the anvils and the blades are formed as electrodes to which high-frequency energy can be applied to melt the first and second tubes at the separation points before and/or during separation of the tubes. The moving unit brings the separated ends of the first and second tubes into abutment in a molten state.

### CROSS-REFERENCE TO RELATED APPLICATIONS

This application is the United States national stage entry of International Application No. PCT/EP2023/083746, filed on Nov. 30, 2023, and claims priority to German Application No. 10 2022 132 053.7, filed on Dec. 2, 2022. The contents of International Application No. PCT/EP2023/083746 and German Application No. 10 2022 132 053.7 are incorporated by reference herein in their entireties.

### FIELD

The present disclosure relates to a device and a method for connecting tubes made of plastic, in particular tubes connected to blood bags.

### BACKGROUND

US 2020/0 047 423 A1 discloses a device which uses a so-called wafer, which is heated to approximately 300 °C by supplying electrical energy, in order to separate two blood bag tubes which are to be connected. Due to the heating of the wafer, the two tubes to be connected are melted at their respective separation points. The molten ends of the tube portions to be connected are then brought together in order to connect the tubes to each other. A disadvantage of this technique is that the wafers are expensive due to the conductor track included therein and are to be disposed of after a connection process due to the high heating and the subsequent cooling process. Accordingly, a connection process is associated with high costs and material use.

EP 4 035 723 A1 attempts to reduce the costs for a connection process by replacing the wafer with a cutting element which is heated by bringing it into contact with a heating arrangement. By using this procedure, the costs for a connection process can be reduced. However, it is also necessary to dispose of the cutting element after the separation process so that a connection process is still associated with cost and material use.

### SUMMARY

It is an object of the present disclosure to provide an improved device and an improved method for connecting tubes made of plastic, in particular tubes connected to blood bags.

A device for connecting tubes made of plastic, in particular tubes connected to blood bags, comprises a first clamp and a second clamp each having two opposite jaws for clamping a first tube and a second tube. The tubes can be clamped by the clamps and connecting of the tubes is also executable in a fluid-carrying state of the tubes.

The device further comprises a first separation unit and a second separation unit each having an anvil and a blade for separating the first and second tubes at a respective separation point. By using the anvil and the blade, the two tubes can be separated without the need of fixing them by means of an additional clamp.

The device further comprises a moving unit adapted to move at least one of the first clamp or the second clamp such that separated ends of the first and second tubes clamped in the clamps can be brought into axial alignment and then into abutment or contact. Accordingly, the separated ends of the tubes to be connected and clamped in the clamps can be brought into abutment and are thereby joined together.

The jaws of the first and second clamps and/or the anvil and the blade of the first and second separation units are formed as electrodes. High-frequency energy can be applied to the electrodes to melt the first and second tubes in the region of the separation point before and/or during separation of the tubes. It should be noted that “molten” means that the tubes 10, 12 made of plastic, in particular PVC, are already softened to such an extent that they melt together on contact and are connected to each other in a cooled state.

The closed electrodes with the tube arranged therebetween as dielectric thus form a capacitor. By applying high-frequency energy to the capacitor, an electric field is generated between the electrodes and the tube is heated such that it is melted. Accordingly, a separation process of the first and second tubes can be carried out in a simple manner.

According to the present disclosure, the jaws of the first or second clamp can be formed as electrodes. In this case, the first and second separation units are arranged adjacent to the first and second clamp, respectively, so that a separation point of the first and second tubes is still sufficiently molten on separation. Alternatively, the anvil and the blade of the first and second separation units can also be formed as electrodes to melt the separation point of the tube. However, it is also conceivable that both the jaws as well as the anvil and the blade are formed as electrodes to which high-frequency energy is applied.

The moving unit is adapted to bring the clamped and separated ends of the first and second tubes into abutment in a molten state. Accordingly, the separated and clamped ends of the first and second tubes are joined to each other with an exact fit, so that after cooling of the tubes a connection between the first and second tubes is established.

Advantageously, the clamps, the separation units and the moving unit can each be driven by electromotive force. Accordingly, a simple control of the individual components can be implemented e.g. by control by means of a control unit, such as a microcontroller.

Furthermore, it is advantageous if the high-frequency energy has a frequency between 25 and 45 MHz. This frequency range is especially suitable for melting by means of high-frequency energy. For medical applications, in particular a frequency of 40.68 MHz±5 kHz has proven to be advantageous for melting blood bag tubes.

Preferably, the blades each have a width in a direction transverse to the axial direction of the first and second tubes, which corresponds to at least one diameter of the first and second tubes.

Accordingly, the tubes can be reliably and completely separated.

According to one aspect, the device according to the disclosure can comprise a high frequency generator adapted to apply high-frequency energy to the electrodes. For this purpose, the high frequency generator can comprise a coil so that an oscillating circuit is formed together with the capacitor formed by the electrodes. Alternatively, the coil can also be arranged in the device connected to the electrodes. The high frequency generator can in particular be connected to the electrodes by means of a coaxial cable. In case both the jaws as well as the blade and the anvil can be applied with high-frequency energy, the high frequency generator can comprise a plurality of outputs or a plurality of high frequency generators can be used. It is also conceivable that different frequencies are used for the jaws and for the blade and the anvil. Particularly preferably, the high frequency generator is communicatively connected to a control unit of the device so that an application of high-frequency energy to the electrodes can be appropriately controlled.

According to an advantageous aspect of the disclosure, the jaws of the first and second clamps can be formed as electrodes to which high-frequency energy can be applied by the high frequency generator. The high frequency generator can then be adapted to apply high-frequency energy to the jaws of the first and second clamps before and/or while the clamped and separated ends of the first and second tubes are brought into abutment. Accordingly, high-frequency energy can again be applied to the separated ends of the tubes which are still held in the clamp so that they are melted again. In this way, a reliable and secure connection of the two tubes can be achieved.

Advantageously, the moving device can be adapted to, before and/or while the clamped and separated ends are brought into abutment, interrupt the bringing into abutment of the clamped and separated ends. Consequently, high-frequency energy can again be applied to the jaws of the clamps to melt the separated ends clamped in the clamps for a sufficient time so that a reliable connection of the two tubes is obtained.

It is advantageous when the first and second clamps are adapted to, after the bringing into abutment and cooling of the separated ends brought into abutment, to continue to clamp the ends. In this case, the moving unit can be adapted to move the closed clamps away from each other in an axial direction of the tubes. In this way, it is achieved that the connection point between the connected tubes is opened so that a fluid flow is reliably enabled. In addition, the connection point can be checked with respect to its tensile strength.

The moving unit can preferably comprise two platforms arranged parallel to each other in a plane. At least one of the platforms can be movable in the plane. The first clamp and the second separation unit can then be arranged on a first platform and the second clamp and the first separation unit can be arranged on a second platform. Accordingly, the two clamps can be moved relative to each other by means of a simple construction in order to bring the two separated ends of the first tube and the second tube clamped in the clamps into axial alignment and then into abutment.

Advantageously, the blades can each have a straight portion parallel to an abutment surface of the anvil. Accordingly, the design of the blade as an electrode can be improved. In addition, the tube portion to be separated, which is not clamped in the clamp, can be welded so that a separation and thus also a connection of fluid-carrying tubes is enabled.

Furthermore, it is advantageous when the blades each have an inclined portion inclined away from the clamp. Accordingly, an even more reliable welding of the tube portion to be separated is achieved.

According to an advantageous aspect, the jaws, in a closed state, can each form protrusions around the tube clamped therein in a direction in which the clamped and separated ends of the tubes are to be brought into abutment. The protrusions thus function as a punch around the clamped tube. Accordingly, an even better bringing into abutment and thus connection of the two separated and clamped ends of the first and second tubes is achieved.

Advantageously, an abutment surface of the anvil and a clamping surface of one of the jaws can be arranged in alignment with respect to the tube to be separated, i.e. an axial direction of the tube, during separation of the first and second tubes. Since the tube is melted in the region of the separation point during separation, it can already be damaged by a slight bending, so that an interior of the tube is contaminated. By arranging the abutment surface of the anvil and the clamping surface of one of the jaws, which is arranged on the side of the anvil, in alignment, this damage and the associated contamination can be prevented. Accordingly, in this way, a sterile connection of the two tubes can be ensured even more reliably.

It has been shown that it is advantageous when a first and a second guiding unit for guiding the first and second tubes are provided. Accordingly, the tubes are properly guided to the clamps and the separation units without being rotated or bent or being arranged in another disadvantageous manner.

Furthermore, it is also advantageous when a first holding unit for holding a first blood bag connected to the first tube and a second holding unit for holding a second blood bag connected to the second tube are provided. The holding units can be formed by two support surfaces or by pins or hooks on which blood bags can be suspended. Accordingly, an careless dropping of the blood bags can be prevented.

According to an exemplary aspect, the device can comprise a cover, which is in particular transparent, and a sensor adapted to detect a closed state of the cover. Connecting of the first and second tubes can only be executable when the sensor detects a closed state of the cover. Accordingly, a contamination of the tubes to be connected is reliably prevented and an operator of the device is protected from carelessly engaging in the moving components of the device. In addition, a connection process can be monitored by the operator through a transparent cover.

The device can advantageously comprise an input unit for detecting a type of the tubes to be connected and/or an operator of the device. The input unit can be formed in a variety of ways, e.g., by a keyboard, a touch screen, a barcode or QR code reader, an RFID scanner or a fingerprint scanner. Accordingly, high-frequency energy can be applied to the electrodes in accordance with the tubes to be connected so that a proper melting is achieved. In addition, the person who has carried out a connection process can be documented.

A method according to the disclosure for connecting tubes made of plastic, in particular tubes connected to blood bags, initially comprises a step for clamping a first tube and a second tube in a first clamp and a second clamp each having two opposite jaws. The tubes can be clamped by the clamps and connecting of the tubes is also executable in a fluid-carrying state of the tubes.

The method additionally comprises a step of separating the first and second tubes at a respective separation point by means of a first separation unit and a second separation unit each having an anvil and a blade. By using the anvil and the blade, the two tubes can be separated without the need of fixing them by means of a second clamp.

Subsequently, a step of moving at least one of the first clamp or the second clamp such that separated ends of the first and second tubes clamped in the clamps are brought into axial alignment and then into abutment is executed in the method. Accordingly, the separated ends of the tubes to be connected and clamped in the clamps can be brought into abutment and joined together.

Before and/or during separation of the tubes, a step of applying high-frequency energy to the jaws of the first and second clamps and/or the anvil and the blade of the first and second separation units formed as electrodes to melt the first and second tubes in the region of the separation point is executed. The jaws of the first and second clamps and/or the anvil and the blade thus function as capacitor plates and the tube located therein functions as dielectric. Consequently, by applying high-frequency energy, the tube is melted so that a proper and sterile separation is achieved. Consequently, in contrast to the state of the art, it is not necessary to heat the separation unit and in particular the blade itself so that it is not to be disposed of after separation.

Subsequently, during the step of moving, the clamped and separated ends of the first and second tubes are brought into abutment in a molten state. Accordingly, the separated and clamped ends of the first and second tubes are joined to each other with an exact fit, so that after cooling a reliable connection between the first and second tubes is established.

Preferably, the method can comprise, before clamping the first tube, a step of providing a blood bag connected to the first tube, and before clamping the first tube or at least before clamping the second tube, a step of providing a blood bag connected to the second tube. The blood bags can be held by a holding unit so that the method can be carried out reliably in an appropriate manner.

Particularly preferably, when carrying out the method, at least one of the first tube or the second tube can be connected to a branch piece or a Y-branch to which the first or the second blood bag and a further blood bag are connected via respective tubes. Accordingly, it is possible, for example, to provide a blood bag for taking blood from a blood donor with only one tube and a needle attached thereto. After the blood sample has been tested for its usability and found to be suitable, it can be separated into its blood components, for example, by centrifugation. Subsequently, the tube of the blood bag comprising the separated blood components can be separated from the needle and connected to a tube to which at least two further blood bags are connected via respective tubes via a branch piece. Subsequently, two of the blood components, for example, plasma and buffy coat, can be transferred from the original blood bag into the further blood bags. This procedure avoids plastic waste which occurs when a taken blood sample is unusable or is not subsequently released by the donor.

In summary, it can be stated that the device according to the disclosure and the method according to the disclosure enable a reliable connection of two tubes made of plastic without separation elements, for example, wafers or blades, having to be disposed of after a separation process. Accordingly, the ongoing costs for a connection process can be reduced.

### BRIEF DESCRIPTION OF THE DRAWINGS

Embodiments of the present disclosure are described below with reference to the attached drawings. In the drawings:

FIG. 1 shows an overall view of a device for connecting tubes according to the present disclosure;

FIG. 2 shows a detailed view of clamps, separation units, a moving unit and two tubes to be connected in the device;

FIG. 3 shows a schematic view of electrodes for heating a tube located therein and the high frequency generator connected thereto; and

FIGS. 4 to 11 show a detailed view of the clamps, the separation units, the moving unit and the two tubes to be connected in the device for connecting tubes during a connection process.

### DETAILED DESCRIPTION

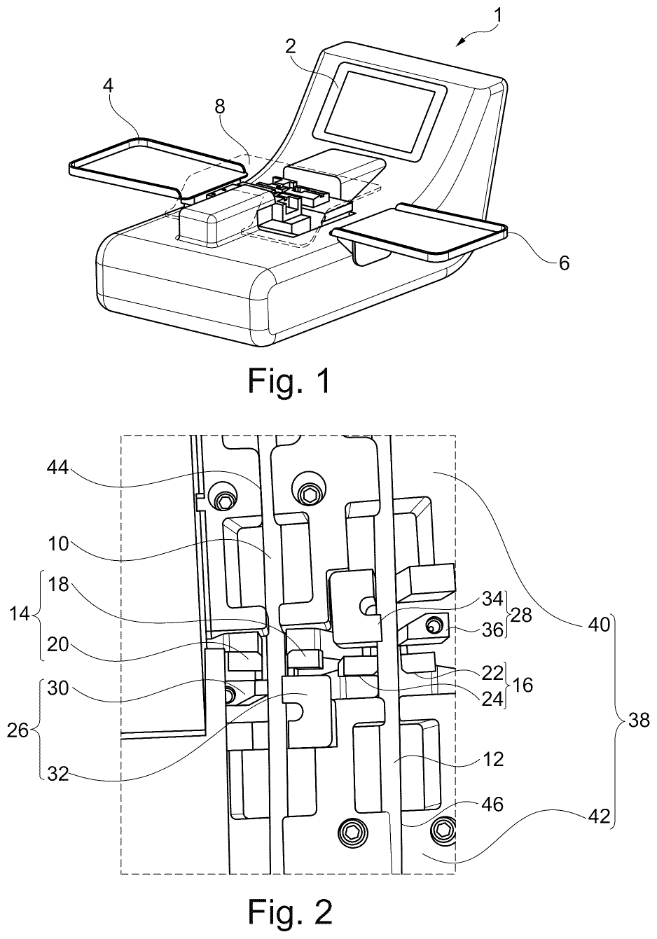

FIG. 1 shows a device 1 according to the disclosure for connecting tubes made of plastic. The device 1 is described below with respect to use with blood bags. The tubes connected to the blood bags are made of PVC. However, the device 1 is also suitable for connecting other tubes which are not made of PVC but can be heated by means of high frequency.

As shown in FIG. 1, the device 1 comprises an input unit 2, a first holding unit 4 for holding a first blood bag (not shown), a second holding unit 6 for holding a second blood bag (not shown) and a cover 8.

The input unit 2 is adapted to detect a type of the tubes to be connected and/or an operator of the device 1. In the present embodiment, the input unit 2 is formed as a touch screen. However, the input unit 2 can also be formed by a keyboard, a barcode or QR code reader, an RFID scanner or a fingerprint scanner or by a combination of these units. Accordingly, an operator of the device can input the type of the tubes to be connected into the device 1 so that the connection process is appropriately executable. In addition, it is documented which operator has executed the connection process. This is necessary when it turns out that a blood sample has been contaminated.

In addition, the device 1 comprises the holding units 4 and 6 for holding a first and a second blood bag. In the present embodiment, the holding units 4 and 6 are formed by two supports including a rim. However, the holding units 4 and 6 can also be formed by pins or hooks on which the blood bags can be suspended. Consequently, an accidental dropping of the blood bags by an operator is avoided.

In addition, the device 1 comprises a cover 8, which is preferably transparent. In addition, the device 1 comprises a sensor (not shown) which can detect a closed state of the cover 8. A control unit of the device 1 connected to the sensor is adapted such that it only starts a connection process when a closed state of the cover 8 is detected. Accordingly, a contamination of the tubes to be connected, e.g. by a contact of the separated ends of the two tubes, can be avoided. In addition, it can be avoided that an operator carelessly engages in the device 1. Due to the transparent design of the cover 8, the connection process can be observed.

After the blood bags are arranged on the holding units 4 and 6, the tubes 10, 12 to be connected can be inserted into the device 1 with the cover 8 opened. FIG. 2 shows the arrangement of a first tube 10 and a second tube 12, which are to be connected, in the device 1. It should be noted that the first tube 10 is connected to the first blood bag on the first holding unit 4 and the first blood bag is located at the top in the example shown in FIG. 2. Consequently, the second blood bag is arranged at the bottom in the example shown in FIG. 2.

As shown in FIG. 2, the device 1 comprises a first clamp 14 and a second clamp 16. The first clamp 14 and the second clamp 16 are each formed by two opposite jaws 18 and 20, and 22 and 24, respectively, which have flat clamping surfaces. The clamps 14, 16 are arranged such that they receive and clamp the proximal ends, i.e. the ends connected to the blood bags. Consequently, a fluid flow from the blood bag through the tubes 10, 12 can be interrupted by the clamps 14, 16.

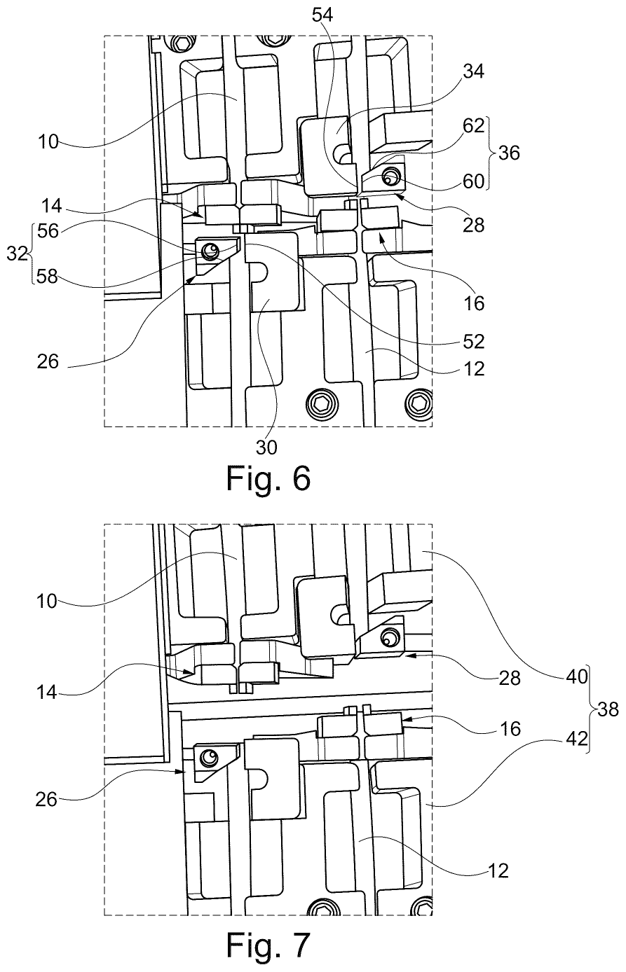

The device 1 additionally comprises a first separation unit 26 and a second separation unit 28 each formed by an anvil 30 and 34, respectively, and a blade 32 and 36, respectively. The two tubes 10 and 12 are separated by the separation units in that the blades 32 and 36, respectively, abut against abutment surfaces 52, 54 (see FIG. 6) of the anvil 32 to 34. A precise design of the blades 32, 36 is described below with reference to FIG. 6.

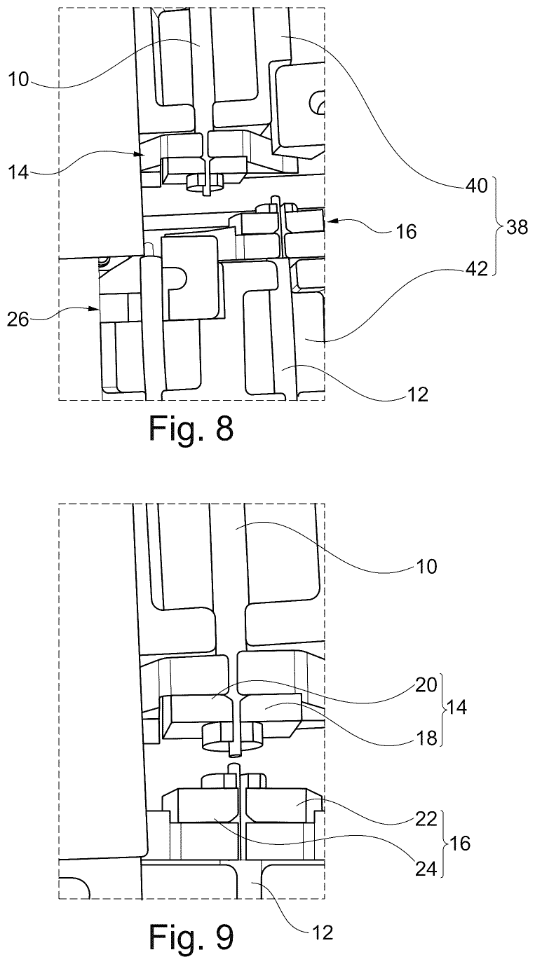

The device 1 further comprises a moving unit 38 formed by a first platform 40 and a second platform 42. The two platforms 40, 42 are arranged parallel to each other in a plane. The first clamp 14 and the second separation unit 28 are arranged on the first platform 40 and the second clamp 16 and the first separation unit 26 are arranged on the second platform 42. Accordingly, as described below with reference to FIGS. 7 and 8, a bringing together or bringing into abutment can be carried out in a simple manner by shifting the two platforms 40, 42 relative to each other.

The first and second platforms 40, 42 comprise a first guiding unit 44 and a second guiding unit 46, respectively, for guiding the first and second tubes 10, 12 to the clamps 14, 16 and the separation units 26, 28. The first and second guiding units 44, 46 can be formed by groove sections in the first and second platforms 40, 42.

The clamps 14, 16, the separation units 26, 28 and the moving unit 38, i.e. at least one of the two platforms 40, 42, can be driven or moved by electromotive force and an operation is controlled by a control unit (not shown) of the device 1 by executing a corresponding computer program. In addition, the control unit is connected to further units, such as the input unit 2, and sensors, such as the sensor for detecting a closed state of the cover 8, in order to properly control a connection process.

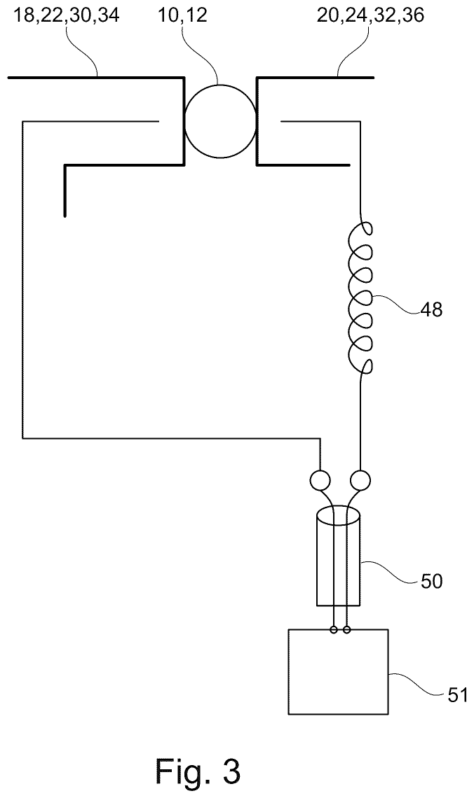

For separating and connecting the first tube 10 and the second tube 12 to each other, the tubes 10, 12 must be melted before and/or during the separation process. For this purpose, the jaws 18 to 24 and/or the anvils 30, 34 and the blades 32, 36, as shown in FIG. 3, are formed as electrodes to which high-frequency energy can be applied. The first tube 10 and the second tube 12, respectively, are arranged between the electrodes. Accordingly, the tubes 10, 12 form a dielectric between the electrodes so that a capacitor is obtained. When high-frequency energy is applied, an electric field is generated between the electrodes, which in turn heats the dielectric located between the electrodes, i.e. the tubes 10, 12, and melts it when sufficient heat is supplied. The tubes 10, 12 can then be separated in a simple manner and subsequently brought into abutment in a molten state. It should be noted that “molten” means that the tubes 10, 12 made of plastic, in particular PVC, are already softened to such an extent that they fuse together on contact and are connected to each other in a cooled state.

As shown in FIG. 3, a coil 48 can be connected to the electrodes in order to form an oscillating circuit. The coil 48 can be connected to the electrodes in the device 1. The oscillating circuit in the device 1 can then be connected or is connected to a high frequency generator 51 via a coaxial cable 50. However, it is also conceivable that the coil 48 is arranged in the high frequency generator 51. It should be mentioned that high-frequency energy can be applied simultaneously to the electrodes for heating the separation points of the first tube 10 and the second tube 12, respectively, in order to ensure that the tubes 10 and 12, respectively, are sufficiently molten when bringing them into abutment. This can be enabled by a plurality of parallel outputs in the high frequency generator 51 or by using a plurality of high frequency generators 51.

The following configurations are conceivable for the design of the electrodes. On the one hand, the anvils 30, 34 and the blade 32, 36 can be formed as electrodes. The tubes 10 and 12 are then clamped and heated between the anvils 30, 34 and the blades 32, 36 during a separation process. Subsequently, the blades 32, 36 are driven further and then abut against the abutment surfaces 52 and 54, respectively (see FIG. 6) of the anvils 30, 34 so that the tubes 10, 12 are separated. The separated ends which are to be connected to each other and are clamped in the clamps 14, 16 are then brought into abutment in the molten state so that after cooling of the tubes 10, 12 a connection of the two tubes 10, 12 is obtained.

Alternatively, the jaws 18 to 24 can be formed as electrodes to heat the tubes 10 and 12 clamped therein. The separation units 26, 28 are then arranged adjacent to the jaws 18 to 24, i.e. the clamps 14 and 16, so that a separation point is still sufficiently molten. This embodiment offers the advantage that the ends of the tubes 10 and 12 clamped and separated in the clamps 14 and 16 can also still be heated before and/or during bringing into abutment.

In addition, it is also conceivable that both the jaws 18 to 24 as well as the anvils 30, 34 and the blades 32, 36 are formed as electrodes so that, for example, heating of the tubes 10, 12 during the separation process is carried out by the separation units 26, 28. However, heating of the tubes 10, 12 can also be carried out during the separation process by the separation units 26, 28 and the clamps 14, 16. This can be necessary especially in the case of tubes 10, 12 with high wall thicknesses. Before and/or during bringing into abutment, the separated ends of the tubes 10, 12 can then be heated again by the clamps 14, 16 so that a reliable connection between the two tubes 10, 12 is obtained.

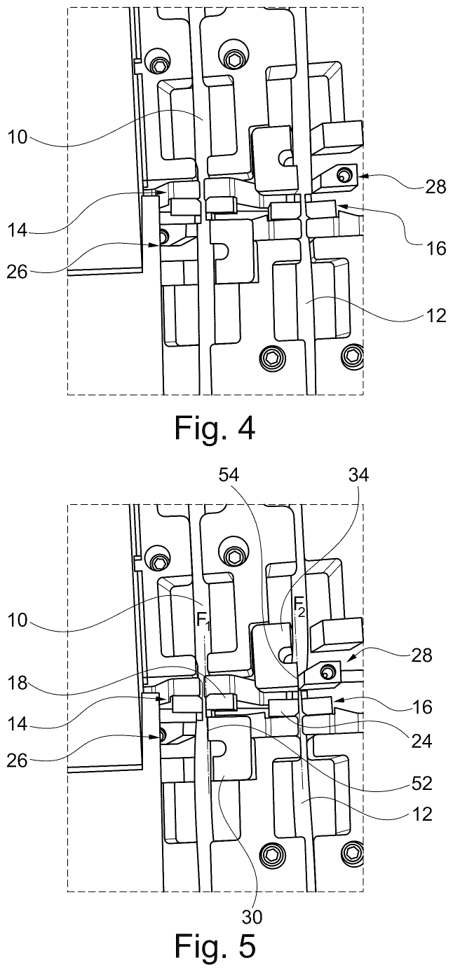

After the tubes 10, 12 are arranged in the device 1, i.e. the tubes 10, 12 are guided in the guiding units 44 and 46, respectively, to the clamps 14 and 16, respectively, and the separation units 26, 28 and a closed state of the cover 8 is detected, a connection process is started by the control unit of the device. A connection process using the above-described device 1 for connecting tubes 10, 12 made of plastic is described below with reference to FIGS. 4 to 11.

As shown in FIG. 4, the clamps 14, 16 are first closed and the first and the second tube 10, 12, respectively, are clamped so that a fluid flow is interrupted. The separation units 26, 28 are still opened in FIG. 4.

As shown in FIG. 5, the jaw 18 and the anvil 30 can preferably be arranged before the separation process such that an inner surface of the jaw 18 and an abutment surface 52 of the anvil 30 are arranged in alignment with respect to an axial direction of the tube 10 (see alignment line F1 in FIG. 5). Hereby, a bending during the separation process can be avoided so that the tube 10 is not damaged in a molten state. In the same way, the jaw 24 and the anvil 34 can be arranged such that an inner surface of the jaw 24 and an abutment surface 54 of the anvil 34 are arranged in alignment with respect to an axial direction of the tube 12 (see alignment line F2 in FIG. 5).

In FIG. 6, the first and the second separation unit 26 and 28 are then also closed. Subsequently, the tubes 10 and 12 are heated in the region of a separation point at which they are to be separated. The heating is carried out, as described above, by applying high-frequency energy to the clamps 14 and 16 or to the separation units 26 and 28. Alternatively, high-frequency energy can also be applied to the clamps 14, 16 and the separation units 26, 28.

As shown in FIG. 6, the blades 32 and 36 preferably have a straight portion 56 and 60, respectively, parallel to the abutment surfaces 52 and 54, respectively. Accordingly, the blades 32, 36 can be suitably formed as electrodes in order to appropriately heat the tubes 10, 12 arranged in the separation units 26, 28. In addition, the blades 32 and 36 can each have an inclined portion 58 and 62, respectively. The inclined portions 58, 62 are inclined away from the clamps 14 and 16, i.e. in the direction of the distal ends of the tubes 10, 12. By the cooperation of the straight portions 52, 56 with the inclined portions 54, 58, the ends of the tubes 10, 12 to be separated can be welded in the molten state. Accordingly, an outflow of a fluid from the separated ends of the tubes 10, 12 can be prevented. The device 1 is thus suitable for connecting two fluid-carrying tubes 10, 12.

After the tubes 10, 12 are separated at a respective separation point, the clamps 14, 16 and the separation units 26, 28, as shown in FIG. 7, are spatially separated from each other by moving the first and second platforms 40 and 42 of the moving unit 38 away from each other in the axial direction of the tubes 10, 12. It should be noted that this can be carried out by moving at least one of the platforms 40 or 42 or by moving both platforms 40 and 42. Preferably, in this process, the separation units 26 and 28 are also closed so that a proper separation of the tubes 10, 12 is ensured.

Subsequently, one of the platforms 40 or 42 or both platforms 40 and 42 are displaced in a direction transverse to the axial direction of the tubes 10, 12 in order to bring the two separated ends of the tubes 10, 12 clamped in the clamps 14, 16 into axial alignment. As already mentioned, this process can be carried out in a simple manner since the moving unit 38 comprises the first platform 40 and the second platform 42 and the first clamp 14 and the second separation unit 28 are arranged on the first platform 40 and the second clamp 16 and the first separation unit 26 are arranged on the second platform 42. Consequently, the two tubes 10, 12 are arranged antiparallel with respect to their flow direction from a blood bag to an outlet of the tubes 10, 12. Accordingly, the tubes can be brought into axial alignment by a simple displacement in a direction transverse to the axial direction of the tubes 10, 12.

After the two tubes 10, 12 have been brought into axial alignment, they are moved towards each other in the axial direction, as shown in FIG. 9, in order to bring them into abutment or contact. As already mentioned above, the jaws 18, 20, 22, 24 of the clamps 14, 16 can be formed as electrodes and can accordingly again be applied with high-frequency energy before and/or during the bringing into abutment so that the ends of the tubes 10, 12 clamped and separated in the clamps 14, 16 are melted again. In this case, the moving device 38 can be adapted to interrupt the bringing into abutment of the clamped and separated ends. Consequently, high-frequency energy can be applied to the clamps 14, 16 for an appropriate time so that the separated ends of the two tubes 10, 12 are sufficiently molten. Accordingly, a proper connection between the two tubes 10, 12 is ensured even more reliably.

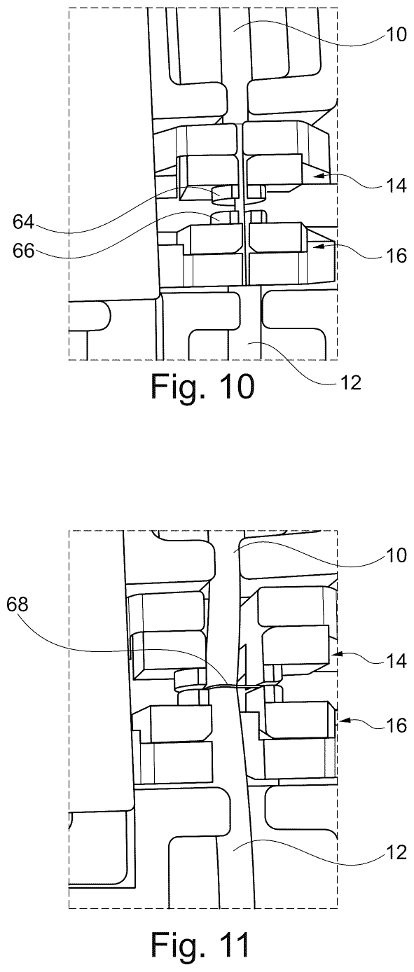

In FIG. 10, the bringing into abutment of the two separated and clamped ends of the tubes 10, 12 is shown. Preferably, the clamps 14, 16, in a closed state, form protrusions 64, 66 around the ends of the tubes 10, 12 clamped and separated therein in a direction in which the ends are to be brought into abutment. The protrusions 64, 66 can be cylindrical or cuboid and function as a punch in order to ensure a reliable bringing into abutment or contact of the separated and molten ends.

After the two ends are brought into abutment and are cooled, the two clamps 14, 16, in a closed state, can be moved away from each other in the axial direction of the tubes 10, 12 (not shown in the drawings). In this way, a connection of the two tubes 10, 12 can be tested for its tensile strength. In addition, a connection point 64 (see FIG. 10) is opened by this moving apart so that a fluid flow between the two tubes 10, 12 is enabled.

Subsequently, the clamps 14, 16 are opened and the tubes 10, 12 connected at a separation point 68 can be removed from the device 1.

Consequently, the device 1 for connecting tubes made of plastic according to the present disclosure is capable of connecting two tubes 10, 12 to each other without generating wear material. Accordingly, the running costs for a connection process can also be significantly reduced.

Particularly preferably, when carrying out the connection process, for example, the first tube 10 can be connected at its one end to a blood bag in which blood from a blood donor is located. The other end of the tube 10 is then connected to a needle by means of which the blood was taken from the blood donor. The second tube 12 is connected to a branch piece or a Y-branch to which at least two further blood bags, so-called satellite bags, are connected. After the blood sample has been tested for its usability and found to be suitable, it can be separated into its blood components, for example, by centrifugation. Subsequently, the tube 10 can be separated from the needle and connected to the tube 12. Subsequently, two of the blood components, for example, plasma and buffy coat, can be transferred from the original blood bag into the further blood bags. This procedure avoids plastic waste which occurs when a taken blood sample turns out to be unusable or is not subsequently released by the donor. In addition, a blood donation process is significantly simplified since a blood donor or a blood donation personnel has to handle less material.

As already mentioned above, the present disclosure has been described with respect to tubes 10, 12 which are connected to blood bags. However, the disclosure can also be applied to other tubes which can be heated by means of high frequency. In addition, the disclosure can also be used in other fields, for example, pharmacy, chemistry and biology.

### LIST OF REFERENCE SIGN

1 device for connecting tubes
2 input unit
4 first holding unit
6 second holding unit
8 cover
10 first tube
12 second tube
14 first clamp
16 second clamp
18 jaw
20 jaw
22 jaw
24 jaw
26 first separation unit
28 second separation unit
30 anvil
32 blade
34 anvil
36 blade
38 moving unit
40 first platform
42 second platform
44 first guiding unit
46 second guiding unit
48 coil
50 coaxial cable
51 high frequency generator
52 abutment surface
54 abutment surface
56 straight portion
58 inclined portion
60 straight portion
62 inclined portion
64 protrusion
66 protrusion
68 connection point

1.-17. (canceled)

18. A device for connecting a first tube and a second tube, the device comprising:
a first clamp having opposing first jaws for clamping the first tube;
a second clamp having opposing second jaws for clamping the second tube;
a first separation unit having a first anvil and a first blade for separating the first tube at a first separation point to form a first separated end;
a second separation unit having a second anvil and a second blade for separating the second tube at a second separation point to form a second separated end;
a moving unit configured to move at least one of the first clamp and the second clamp such that the first separated end of the first tube and the second separated end of the second tube are positioned for axial alignment and abutment; and
electrodes configured to receive high frequency energy to melt the first tube at the first separation point during separation of the first tube and melt the second tube at the second separation point before during separation of the second tube,
the electrodes comprising:
the opposing first jaws and the opposing second jaws, and/or
the first anvil, the second anvil, the first blade, and the second blade, and
the moving unit adapted to bring the first separated end of the first tube and the second separated end of the second tube into abutment in a molten state.

19. The device according to claim 18, further comprising:
a high frequency generator adapted to apply high-frequency energy to the electrodes.

20. The device according to claim 19, wherein:
the electrodes comprise the opposing first jaws and the opposing second jaws,
the high frequency generator is configured to apply high-frequency energy to the opposing first jaws and the opposing second jaws before and/or while the first separated end of the first tube is in abutment with the second separated end of the second tube.

21. The device according to claim 18, wherein:
the moving unit is configured to interrupt relative movement of the first separated end and the second separated end before and/or while the first separated end and the second separated end are brought into abutment.

22. The device according to claim 18, wherein
the first clamp and the second clamp are configured to clamp the first separated end and the second separated end after the first separated end and the second separated end are brought into abutment and cooled, and
the moving unit is configured to move the first clamp and the second clamp away from each other in an axial direction of the first tube and the second tube.

23. The device according to claim 18, wherein:
the moving unit comprises a first platform and a second platform arranged parallel to each other in a plane,
at least one of the first platform and the second platform is movable in the plane,
the first clamp and the second separation unit are arranged on the first platform, and
the second clamp and the first separation unit are arranged on the second platform.

24. The device according to claim 18, wherein:
the first blade has a first straight portion and the first anvil has a first abutment face parallel to the first straight portion, and
the second blade has a second straight portion and the second anvil has a second abutment face parallel to the second straight portion.

25. The device according to claim 18, wherein the first blade and the second blade each have an inclined portion inclined away from the second clamp and the first clamp, respectively.

26. The device according to claim 18, wherein:
the opposing first jaws form a first protrusion around the first tube when the first tube is clamped in the opposing first jaws,
the opposing second jaws form a second protrusion around the second tube when the second tube is clamped in the opposing second jaws, and
the first protrusion and the second protrusion face one another.

27. The device according to claim 18, wherein:
the first anvil comprises a first abutment surface, and the first clamp forms a first clamping surface arranged in alignment with the first abutment surface and with respect to the first tube during separation of the first tube, and
the second anvil comprises a second abutment surface, and the second clamp forms a second clamping surface arranged in alignment with the second abutment surface and with respect to the second tube during separation of the second tube.

28. The device according to claim 18, further comprising a first guiding unit and a second guiding unit for guiding the first tube and the second tube.

29. The device according to claim 18, further comprising:
a first holding unit for holding a first blood bag connected to the first tube; and
a second holding unit for holding a second blood bag connected to the second tube.

30. The device according to claim 18, further comprising:
a cover; and
a sensor configured to detect a closed state of the cover,
wherein the device is operable to connect the first tube and the second tube only when the sensor detects the closed state of the cover.

31. The device according to claim 18, further comprising an input unit for detecting a type of tubes to be connected and/or an operator of the device.

32. A method for connecting a first tube and a second tube, the method comprising the steps of:
clamping the first tube in a first clamp having opposing first jaws;
clamping the second tube in a second clamp having opposing second jaws;
separating the first tube at a first separation point using a first separation unit having a first anvil and a first blade to form a first separated end;
separating the second tube at a second separation point using a second separation unit having a second anvil and a second blade to form a second separated end;
moving at least one of the first clamp and the second clamp such that the first separated end of the first tube and the second separated end of the second tube are positioned for axial alignment and abutment;
applying high-frequency energy to melt the first tube at the first separation point and to melt the second tube at the second separation point; and
abutting the first separated end in a molten state with the second separated end in a molten state to connect the first tube and the second tube.

33. The method according to claim 32, wherein the step of applying high-frequency energy comprises:
applying high-frequency energy to the opposing first jaws before the step of separating the first tube at the first separation point; and
applying high-frequency energy to the opposing second jaws before the step of separating the second tube at the second separation point.

34. The method according to claim 32, wherein the step of applying high-frequency energy comprises:
applying high-frequency energy to the first anvil and the first blade of the first separation unit during the step of separating the first tube at the first separation point; and
applying high-frequency energy to the second anvil and the second blade of the second separation unit during the step of separating the second tube at the second separation point.

35. The method according to claim 32, further comprising the steps of:
providing a first blood bag connected to the first tube before clamping the first tube in the first clamp; and
providing a second blood bag connected to the second tube before clamping the second tube in the second clamp.

36. The method according to claim 35, wherein at least one of the first tube and the second tube is connected to a branch piece configured to connect to the first blood bag, the second blood bag, and a further blood bag.
